(19) United States
(12) Patent Application Publication
(10) Pub. No.: US 2021/0275872 A1
(43) Pub. Date: Sep. 9, 2021

(54) EXTENSOR APPARATUS PROVIDING DYNAMIC ENGAGEMENT FOR TREATMENT OF HAND SPASTICITY AND RELATED DEVICES AND COMPUTER PROGRAM PRODUCTS

(71) Applicant: RehabilliBuddies, Del Mar, CA (US)

(72) Inventors: Joanna D. Cao, Durham, NC (US); Isabella Elizabeth Watson, Del Mar, CA (US); Martha Grace Farrell, Chapel Hill, NC (US); Lars Patrick Chen, Madison, WI (US); Misbah Ahmad, Waxhaw, NC (US); Jack Patrick Vincent, Tucson, AZ (US)

(21) Appl. No.: 17/196,412

(22) Filed: Mar. 9, 2021

Related U.S. Application Data
(60) Provisional application No. 62/987,127, filed on Mar. 9, 2020.

(51) Int. Cl. A63B 23/16; A61F 5/01; A63B 21/008; A63B 24/00; A63B 21/00; A63F 13/67

(52) U.S. Cl. CPC A63B 23/16; A61F 5/0118; A63B 21/0085; A63B 2022/0094; A63B 21/4019; A63F 13/67; A63B 2024/0093; A63B 24/0087

(57) ABSTRACT

An apparatus for treating spasticity can include a splint configured to receive a patient wrist and hand including a wrist portion of the splint including a moveable closure configured to secure a wrist of the patient to the splint. A hand portion of the splint can be rigidly coupled to the wrist portion, wherein the hand portion can include a hand portion that is shaped to receive a palm of the patient's hand and an inflatable bladder can be coupled to the hand portion, wherein the inflatable bladder can be configured to expand against fingers of the patient's hand during inflation.

### CROSS-REFERENCE TO RELATED APPLICATIONS

The present application claims priority to U.S. Provisional Patent Application No. 62/987,127; filed Mar. 9, 2020 entitled EXTENSOR APPARATUS PROVIDING DYNAMIC ENGAGEMENT FOR TREATMENT OF HAND SPASTICITY AND RELATED DEVICES AND COMPUTER PROGRAM PRODUCTS. The disclosure of which is incorporated herein by reference in its entirety.

### FIELD

This invention relates generally to the field of rehabilitation in general, and more particularly, to the treatment of hand spasticity.

### BACKGROUND

Many people suffering a neurological injury, from stroke, cerebral palsy, brain injury, etc., can have upper extremity impairments. Many have some shoulder and elbow movements, but may be unable to extend their wrist or fingers to grasp an object. This can be due to hypertonicity, which is a condition where the flexor or extensor muscles in the upper extremities are spastic and resist positioning. Dynamic splints have been used to hold joints in certain positions including ones that provide enough force to balance the effects of the increased muscle tone. Such a dynamic splint is discussed, for example, in United States Patent Application Publication No. US2003/0162634.

### BRIEF DESCRIPTION OF THE DRAWINGS

FIG. 1 is a schematic illustration of a first portion of a dynamic extensor apparatus that is configured to provide treatment for spasticity in some embodiments according to the invention.

FIG. 2 is a schematic illustration of the apparatus shown in FIG. 1 further including an inflatable bladder to provide treatment for spasticity in some embodiments according to the invention.

FIG. 3A is a schematic representation of the inflatable bladder inflated to a first volume while in contact with the patient's fingers as treatment for spasticity in some embodiments according to the invention.

FIG. 3B is a schematic representation of the inflatable bladder inflated to a second volume that is greater than the first volume while in contact with the patient's fingers in some embodiments according to the invention.

FIG. 4 is a schematic illustration of a system including a processor circuit, a pump, a valve, and the inflatable bladder configured to provide dynamic extensor treatment for spasticity in some in embodiments according to the invention.

FIG. 5 is a flowchart illustrating operations of processor executable code that provides dynamic extensor treatment for spasticity in some embodiments according to the invention.

FIG. 6 is a schematic illustration of game-play provided on the display of an electronic device via the processor executable code as part of a dynamic extensor spasticity treatment in some embodiments according to the invention.

FIG. 7 is a schematic illustration of a competitive presentation appurtenant to the game-play shown in FIG. 6 as part of a dynamic extensor spasticity treatment in some embodiments according to the invention.

### DETAILED DESCRIPTION OF EMBODIMENTS ACCORDING TO THE INVENTION

Exemplary embodiments of the present disclosure are described in detail with reference to the accompanying drawings. The disclosure may, however, be exemplified in many different forms and should not be construed as being limited to the specific exemplary embodiments set forth herein. Rather, these exemplary embodiments are provided so that this disclosure will be thorough and complete, and will fully convey the scope of the disclosure to those skilled in the art.

As described herein in greater detail, in some embodiments according to the invention, an apparatus can provide a dynamic extensor for treatment of spasticity, such as hand spasticity. In particular, in some embodiments, the dynamic extensor can be an inflatable bladder that can be inflated from a first volume to a second volume (that is greater than the first volume) while the patient's fingers are in contact with the inflatable bladder. The inflatable bladder can be attached to the hand portion of a splint that is secured to the patience wrist and both of which can be rigidly coupled to one another.

In operation, the splint is secured to the patient's wrist and hand so that the inflatable bladder is in contact with the inside surface of the patient's fingers (i.e., the portion of the patient's hand which is subject to spasticity). When the system is activated, the inflatable bladder can be inflated from the first volume, for example the volume of air that is provided within the inflatable bladder in a relaxed state) to a second volume such that the patient's fingers are maintained in contact with the surface of inflatable bladder during inflation to the second volume. Once the inflatable bladder is inflated to the second volume, the patient can attempt to squeeze the inflatable bladder using their fingers thereby causing the pressure inside the inflatable bladder to increase, which can be detected by the system. In response, the system then can deflate the inflatable bladder back to the first volume. This cycle can be repeated a number of times (in some embodiments under the control of the patient) to exercise the patient's ability to manipulate their fingers and thereby provide treatment for hand spasticity.

In some embodiments according to the invention, as described above the deflation may be activated in response to the pressure generated by the patient squeezing the inflatable bladder whereupon the inflatable bladder is deflated back to the first volume (an active mode of operation). In still other embodiments according to the invention, the system can operate in a passive mode whereby once activated the inflatable bladder is cycled between the first volume and the second volume such that the deflation is not triggered as a response to increased pressure applied by the patient's fingers. In further embodiments according to the invention, the system can operate in a gameplay mode where by the patient can be provided with an interactive display on an electronic device, such as a cell phone, and prompted for responses as part of a simulated game which can provide the treatment for hand spasticity described herein. For example, in some embodiments according to the invention, the gameplay mode may provide a representation of a basketball game where by the patient is prompted to shoot baskets or dribble the ball wherein the action is taken in response to squeezing the inflatable bladder. Still further, the gameplay mode can also provide a competitive scoreboard by which different patients can be ranked relative to one another as a competition to further motivate the patients to participate in the hand spasticity treatment. In some embodiments according the invention, the identity of the patients involved in the gameplay is kept confidential using, for example, avatars or pseudonyms.

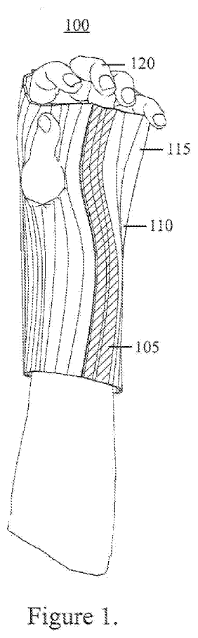

FIG. 1 is a schematic illustration of a first portion of a dynamic extensor apparatus 100 that is configured to provide treatment for hand spasticity in some embodiments according to the invention. According to FIG. 1, the dynamic extensor apparatus 100 is worn over a patient's wrist portion 110 and hand portion 115 of the patient's hand (including the palm) while also allowing the fingers 120 of the patient's hand to extend beyond the apparatus 100. As further shown in FIG. 1, the apparatus 100 includes a rigid splint 105 which extends along the length of the patients arm to rigidly couple the wrist portion 110 to the hand portion 115. It will be understood that the illustration of FIG. 1 is conceptual in nature and is not to be construed as being limited to a particular material or configuration.

Still further, the apparatus 100 can include other items which are not shown in FIG. 1 and may be fabricated from a variety of materials including plastics, woven fabrics, and other materials which can allow for prolonged use as well as allowing breathability and moisture resistance. Still further, it will be understood that the splint portion 105 may be separable from the remainder of the apparatus 100 shown in FIG. 1 such that the remainder may be washed without the splint 105 being present. Still further, the apparatus 100 may include closures or other mechanisms that allow the apparatus 100 to be inserted over the patient's arm and secured.

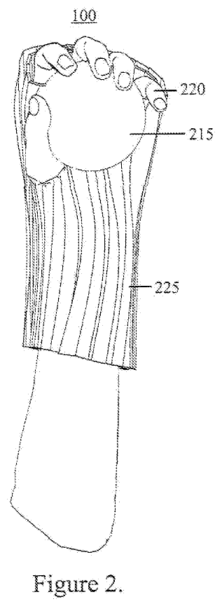

FIG. 2 is a schematic illustration of the apparatus shown in FIG. 1 further including an inflatable bladder 215 to provide treatment for hand spasticity in some embodiments according to the invention. According to FIG. 2, the inflatable bladder 215 is secured to the apparatus 100 (such as the palm portion) to be in contact with the inside surface of the patient's fingers 120 when the apparatus 100 is worn. In operation, the inflatable bladder 215 is configured to remain in contact with the fingers 120 during inflation and deflation of the inflatable bladder 215. For example, when first worn by the patient, the inflatable bladder 215 may be in contact with the inside surface of the patient's fingers 120 despite the fingers 120 being subject to spasticity. Furthermore, as the inflatable bladder 215 is inflated from the initial volume to the final volume, the inside surfaces of the fingers 120 also remain in contact with the inflatable bladder 215 so that the fingers are moved from the spastic position to an open position. Accordingly, during inflation and deflation of the inflatable bladder 215 the fingers 120 of the patient remain in contact with the surface of the inflatable bladder 215. It will be understood that even though the fingers are described herein as being in contact with the inflatable bladder during treatment, momentary or insubstantial lapses in contact may occur during inflation and deflation of the inflatable bladder 215.

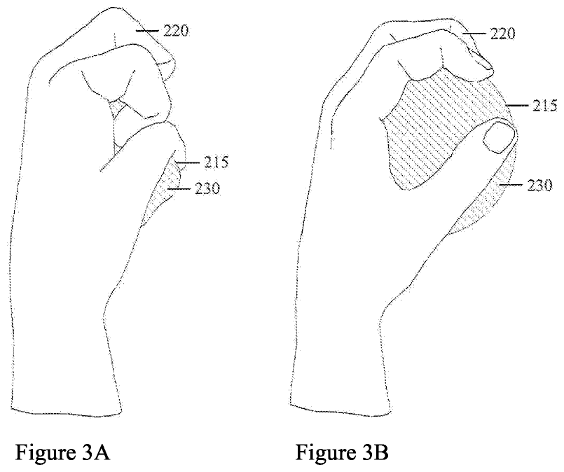

FIG. 3A is a schematic representation of the inflatable bladder 215 inflated to a first volume 230 while in contact with the patient's fingers 120 as treatment for spasticity in some embodiments according to the invention. According to FIG. 3A, the inflatable bladder 215 is in contact with the inside surface of the finger 220 when the inflatable bladder 215 is at the first volume 230. It will be understood that the first volume 230 may be the initial volume at which the treatment begins. Accordingly, the first volume maybe essentially about zero. In still further embodiments according to the invention, the inflatable bladder 215 may be initially partially inflated so as to promote contact with the patient's fingers 120 before treatment for spasticity begins. As further shown in FIG. 3A air is pumped into the inflatable bladder 215 via a tube that is coupled to the inflatable bladder 215.

FIG. 3B is a schematic representation of the inflatable bladder 215 inflated to a second volume 235 that is greater than the first volume 230 while in contact with the patient's fingers in some embodiments according to the invention. According to FIG. 3B, the inflatable bladder 215 is inflated from the first volume 230 to the second volume to 235 using air flow pumped into the inflatable bladder 215 via the tube. Still further, the inside surface of the patient's fingers 120 is maintained in contact with the surface of the inflatable bladder 215. It will be understood that although FIGS. 3A and 3B show a representative finger 220 in contact with the surface of the inflatable bladder 215, all of the fingers of the patient's hand maybe in contact with the inflatable bladder 215 during treatment. Still further, only a subset of fingers 120 maybe in contact with the inflatable bladder 215 whereas other ones of the fingers may not be in contact or are partially in contact with the inflatable bladder 215.

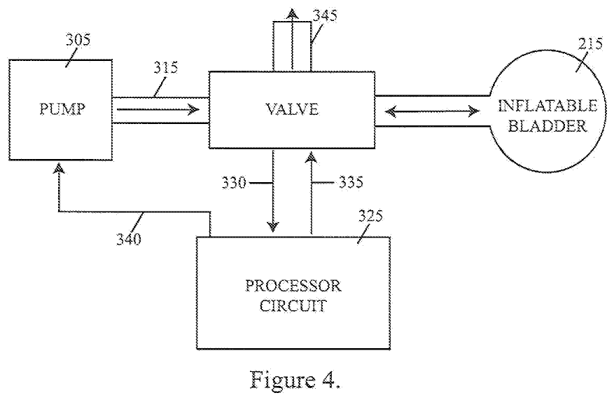

FIG. 4 is a schematic illustration of a system 300 including a processor circuit 325, a pump 305, a valve 345, interconnecting tubes, and the inflatable bladder 215 configured to provide dynamic extensor treatment for hand spasticity in some in embodiments according to the invention. According to FIG. 4, the system 300 operates under the control of the processor circuit 325. Although not explicitly shown the processor circuit 325 is operatively coupled to a memory system or in includes an embedded memory system which provides a media which can store processor executable code that controls operations of the system 300 when executed by the processor circuit 325.

In particular, the processor circuit 325 operates the processor executable code to activate the pump 305 to inflate the inflatable bladder 215 in conjunction with operation of the valve 345. In operation, the pump 305 inflates the inflatable bladder 215 (through control of the valve 345 via signal 330) by pumping air through tubes 315. Still further, the processor circuit 325 also controls operation of the valve 345 so that the air flow from the pump 305 into the inflatable bladder 215 can be interrupted. Still further, the processor circuit 325 can cause the valve 345 to release air from the inflatable bladder 215 via an outlet 345 that may be integrated with the valve 345 or may be separate from the valve 345. For example, the processor circuit 325 can initiate operations by causing the pump 305 to pump air into the inflatable bladder 215 to inflate the inflatable bladder 320 from the first volume 230 to the second volume 235. It will be understood that the air flow through the tubes 315 can be managed by the valve 345 so that air flows one way from the pump 305 into the inflatable bladder 320 but does not flow in the reverse direction. Once the inflatable bladder 215 reaches the second volume 235 the processor circuit 325 can cause the valve 345 to release the air in the inflatable bladder through the outlet 345. Still further, the processor circuit 325 can detect the pressure in the inflatable bladder 215 using a sensor included in the valve 345 or under the control of the valve 345. For example, when the patient squeezes the inflatable bladder 215, the processor circuit 325 can detect the increase in the pressure inside the inflatable bladder 320 using the sensor in the valve 345. Still further, the processor circuit 325 may release the air in the inflatable bladder 215 upon detecting the increase in pressure responsive to the patients squeezing the inflatable bladder 215.

It will be understood that in some embodiments according to the invention, the processor circuit 325 will only release the air in the inflatable bladder 320 in response to pressure exerted by the fingers 120 of the patient if that pressure exceeds a specified threshold value. For example, the threshold value may be set by the system or by the patient to require that a certain amount of minimum pressure should be exerted by the patient during the therapy in order for the bladder 215 to be deflated. In some embodiments according to the invention, the pressure threshold required for deflation of the inflatable bladder in response to pressure exerted by the patient can be set by the patient, the patient's health care professional responsible for patient therapy, or may be automatically adjusted by the system 300 during the course of the patients treatment.

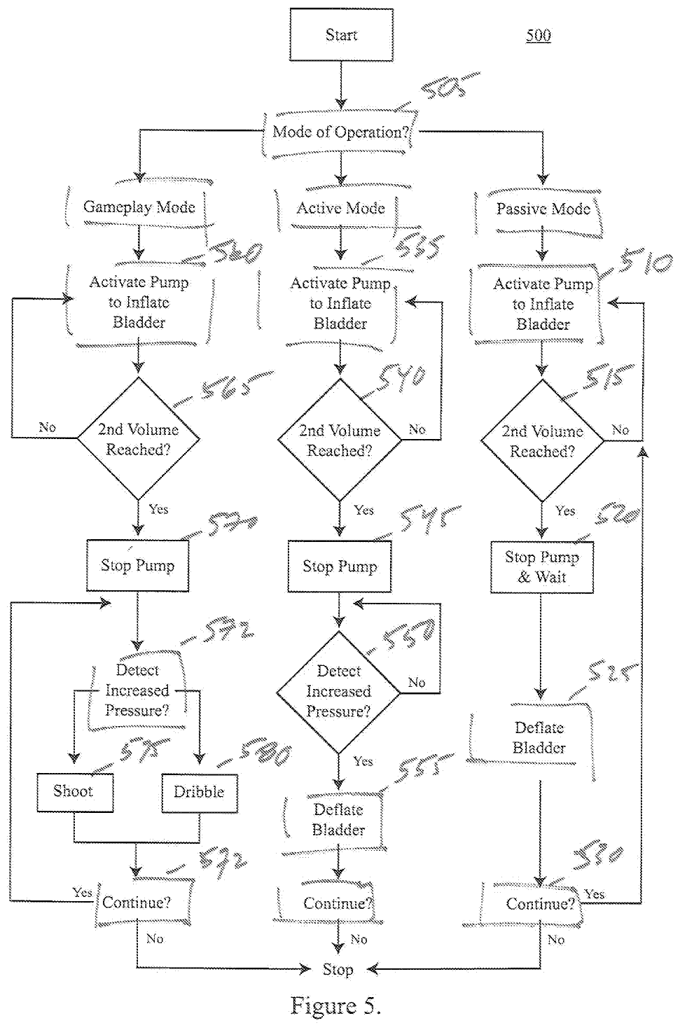

FIG. 5 is a flowchart 500 illustrating operations of processor executable code that provides dynamic extensor treatment for hand spasticity in some embodiments according to the invention. According to FIG. 5, operations begin wherein the processor circuit 325 determines a mode of operation of the apparatus 100 (block 505). If the apparatus 100 determines that the passive mode of operation is to be used, the processor circuit 325 activates the pump (block 510) to inflate the inflatable bladder 215 from the initial volume 230 to the second volume 235. Once the processor circuit 325 determines that the inflatable bladder has been inflated to the second volume (block 515), the processor circuit 325 maintains inflation of the inflatable bladder 325 at the second volume 235 (block 520) until an input is provided either by the system in the passive mode or in response to an input by the patient causing the processor circuit 325 to deflate the inflatable bladder 215 (block 525) whereupon the processor circuit 325 causes the inflatable bladder 215 to be deflated. Operations in the passive mode continue until system operation is ceased (block 530).

If the apparatus 100 determines that the active mode of operation is selected (block 505) the processor circuit 325 begins by inflating the inflatable bladder 215 (block 535) until the second volume 325 is reached (block 540). Once the second volume is reached, the processor circuit 325 stops the pump (block 545) and monitors the pressure in the inflatable bladder 215 to detect an increase in the pressure inside the inflatable bladder 215 indicating that the patient has squeezed the inflatable bladder (block 550) whereupon the processor circuit 325 can deflate the inflatable bladder 215 (block 555).

In some embodiments according to the invention, the apparatus 100 may be configured to require a predetermined threshold value for an increase in the pressure before operations in the active mode can continue. For example, the apparatus 100 may set a particular pressure increase threshold such that the patient should exert a certain minimum pressure on the inflatable bladder 215 before the apparatus 100 will deflate the inflatable bladder 215 to the initial volume 230 (block 555), whereupon operations can continue in the active mode by re inflating the inflatable bladder 215 to the second volume 235.

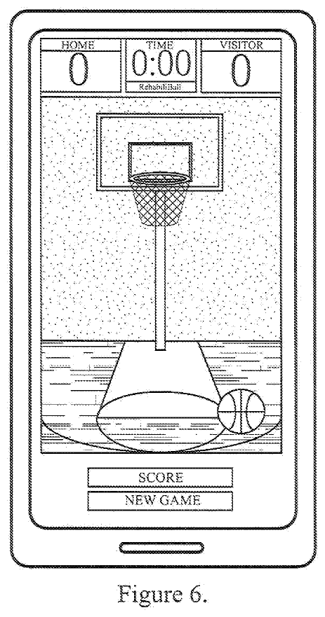

In some embodiments according to the invention, the apparatus 100 can support a gameplay mode of operation whereupon the apparatus 100 activates the pump 325 (block 560) until the inflatable bladder 215 reaches the second volume (block 565). The pump is stopped (block 570) and the patient is then prompted to squeeze the inflatable bladder 215 to simulate an activity in a game, such as a game of basketball. As shown in FIG. 6, different operations can be activated based on the amount of pressure that the patient exerts (block 572) on the inflatable bladder 215. For example, if the patient exerts a relatively low pressure on the inflatable bladder, the gameplay mode may provide feedback to the patient showing the ball being dribbled (block 580), whereas if the pressure exerted on the inflatable bladder 215 is greater than a certain value, the apparatus 100 may provide visual feedback showing the ball being shot towards the basket (bock 575). Accordingly, in some embodiments according to the invention the gameplay mode may provide varied feedback based on varied amounts of pressure exhibited by the patient on the inflatable bladder. Operations in the gameplay mode can continue (block 585) at block 572. It will be understood that any of the modes of operation described herein can be interrupted in response to patient input (such as an indication that the patient is in discomfort). In response to the patient input the bladder 215 can be deflated and further operations can be ceased.

FIG. 6 is a schematic illustration of gameplay provided on the display of an electronic device via the processor executable code as part of a dynamic extensor spasticity treatment in some embodiments according to the invention. According to FIG. 6, the display of an electronic device can be used to provide visual representation of a game in conjunction with the gameplay mode shown in FIG. 5. In particular, in FIG. 6 the electronic device display is shown including a graphic representation of a basketball hoop and a basketball along with time readout providing a time frame for gameplay for the patient. In operation, the ball may be provided on the display whereupon the patient is prompted to squeeze the inflatable bladder and, based on the amount of pressure detected by the apparatus 100 on the inflatable bladder 215, the ball may be shown to perform different operations. For example, if a relatively low pressure is exerted on the inflatable bladder 215, the ball maybe dribbled whereas if a relatively high pressure is exerted on the inflatable bladder 215 the ball maybe shot toward the basket. As further shown in FIG. 6, the total score achieved by the patient during the game may also be displayed.

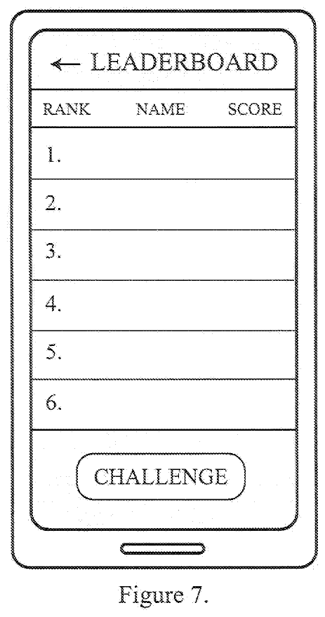

FIG. 7 is a schematic illustration of a competitive presentation appurtenant to the gameplay shown in FIG. 6 as part of a dynamic extensor spasticity treatment in some embodiments according to the invention. According to FIG. 7, a list of scores achieved by different patients may be posted so that all registered patients can view their status relative to other patients. Still further, the competitive presentation may allow patients to challenge certain ones of the other competitors to a head-to-head competition. It will be understood that the identity of each of the competitors may be maintained for patient privacy.

The terminology used herein is for the purpose of describing particular embodiments only and is not intended to be limiting to other embodiments. As used herein, the singular forms “a”, “an” and “the” are intended to include the plural forms as well, unless the context clearly indicates otherwise. It will be further understood that the terms “comprises,” “comprising,” “includes” and/or “including”, “have” and/or “having” when used herein, specify the presence of stated features, integers, steps, operations, elements, and/or components, but do not preclude the presence or addition of one or more other features, integers, steps, operations, elements, components, and/or groups thereof. Elements described as being “to” perform functions, acts and/or operations may be configured to or other structured to do so.

Unless otherwise defined, all terms (including technical and scientific terms) used herein have the same meaning as commonly understood by one of ordinary skill in the art to which various embodiments described herein belong. It will be further understood that terms used herein should be interpreted as having a meaning that is consistent with their meaning in the context of this specification and the relevant art and will not be interpreted in an idealized or overly formal sense unless expressly so defined herein.

“Spasticity” or a “spastic condition” may refer to an involuntary and/or velocity-dependent muscle tone that may cause resistance to movement. Spasticity may result from an injury to the central nervous system or motor neurons. Spasticity may occur as a primary condition such as in degenerative conditions, or as a result of secondary causes such as spinal cord injury, trauma to the brain, or inflammatory conditions including, but not limited to, multiple sclerosis.

As used herein, “patient” and “subject” may be used interchangeably. That is, the patient or subject refers to one in whom prevention and/or treatment of spasticity is needed or desired, as well as any subject prone to spasticity. In some embodiments, the subject is a human; however, a subject of this invention can include an animal subject, particularly mammalian subjects including non-human primates, etc., for veterinary medicine or pharmaceutical drug development purposes or animal healthcare purposes. The subjects may be male or female and may be of any race or ethnicity, including, but not limited to, Caucasian, African-American, African, Asian, Hispanic, Indian, etc. and combinations thereof. The subjects may be of any age, including child, adolescent, adult, and geriatric. In some embodiments, the subject has been diagnosed with spasticity, is at risk for developing a spastic condition, or for whom it is generally desirable to prevent a spastic condition. In some embodiments, the subject is afflicted with or is at risk for developing a degenerative condition, a spinal cord injury, trauma to the brain, and/or an inflammatory condition such as multiple sclerosis. In particular embodiments, the subject is afflicted with or is at risk for developing hand spasticity.

As will be appreciated by one of skill in the art, various embodiments described herein may be embodied as a method, data processing system, and/or computer program product. Furthermore, embodiments may take the form of a computer program product on a tangible computer readable storage medium having computer program code embodied in the medium that can be executed by a computer.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wired, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages, such as a programming language for a FPGA, Verilog, System Verilog, Hardware Description language (HDL), and VHDL. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computer embodiment or offered as a service such as a Software as a Service (SaaS).

Some embodiments are described herein with reference to flowchart illustrations and/or block diagrams of methods, systems and computer program products according to embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall support claims to any such combination or subcombination.

While the foregoing is directed to aspects of the present disclosure, other and further embodiments of the disclosure may be devised without departing from the basic scope thereof, and the scope thereof is determined by the claims that follow.

What is claimed:

1. An apparatus for treating spasticity comprising:
a splint configured to receive a patient wrist and hand including:
a wrist portion of the splint including a moveable closure configured to secure a wrist of the patient to the splint;
a hand portion of the splint rigidly coupled to the wrist portion, the hand portion including a hand portion that is shaped to receive a palm of the patient's hand; and
an inflatable bladder coupled to the hand portion, the inflatable bladder configured to expand against fingers of the patient's hand during inflation.

2. The apparatus of claim 1 wherein the inflatable bladder is configured to inflate to a first inflated volume while in contact with fingers of the patient's hand and configured to inflate to a second inflated volume that is greater than the first volume while in contact with the fingers of the patient's hand during inflation.

3. The apparatus of claim 2 wherein the inflatable bladder is configured to deflate from the second inflated volume to the first volume while in contact with the fingers of the patient's hand during deflation.

4. The apparatus of claim 3 further comprising:
a processor circuit configured to control the inflation and deflation of the inflatable bladder responsive to detecting an increase in pressure in the inflatable bladder responsive to the fingers of the patient's hand compressing the inflatable bladder in an active mode of operation of the apparatus.

5. The apparatus of claim 4 wherein the processor circuit is configured to compare the increase in the pressure to a predetermined value to activate the deflation.

6. The apparatus of claim 3 further comprising:
a processor circuit configured to control the inflation and deflation of the inflatable bladder responsive to detecting an input to the system indicating a passive mode of operation of the system.

7. The apparatus of claim 6 further comprising:
a processor-readable storage medium, having stored thereon process-executable code, that, upon execution by the processor circuit, enables actions comprising:
(a) receiving the input indicating the passive mode of operation;
(b) activating a pump to inflate the inflatable bladder through a valve until reaching the second volume in the inflatable bladder;
(c) activating a switch to bypass the valve to deflate the inflatable bladder to the first volume; and
(d) repeating actions (a) to (c).

8. The apparatus of claim 4 further comprising:
a processor-readable storage medium, having stored thereon process-executable code, that, upon execution by the processor circuit, enables actions comprising:
(a) receiving the input indicating the active mode of operation;
(b) activating a pump to inflate the inflatable bladder through a valve until reaching the second volume in the inflatable bladder;
(c) activating a switch to bypass the valve to deflate the inflatable bladder to the first volume responsive to detecting the increase in the pressure in the inflatable bladder responsive to the fingers of the patient's hand compressing the inflatable bladder; and
(d) repeating actions (a) to (c).

9. The apparatus of claim 8 wherein the process-executable code further enables actions comprising:
recording the increase in the pressure in the inflatable bladder detected in action (c) to provide recorded therapy data; and
providing feedback to the patient including the recorded therapy data.

10. The apparatus of claim 9 wherein the process-executable code further enables actions comprising:
comparing the recorded therapy data for the patient to therapy data for other patients or a therapy data for projected rehabilitation.

11. The apparatus of claim 9 wherein the process-executable code further enables actions comprising:
providing the feedback to the patient as part of game-play; and
providing a comparison between the recorded therapy data for the patient and recorded therapy data for another patient as part of the game-play.

12. The apparatus of claim 11 wherein the feedback to the patient as part of the game-play is provided on an electronic display.

13. A system for treating spasticity comprising:
a splint configured to receive a patient wrist and hand including:
a wrist portion of the splint including a moveable closure configured to secure a wrist of the patient to the splint;
a hand portion of the splint rigidly coupled to the wrist portion, the hand portion including a hand portion that is shaped to receive a palm of the patient's hand;
an inflatable bladder coupled to the hand portion, the inflatable bladder including an input to the inflatable bladder;
a pump configured to generate a gas at an output of the pump; and
a tube having a first opening coupled to the output of the pump and having a second opening coupled to the input to the inflatable bladder.

14. A system for treating spasticity comprising:
a splint configured to receive a patient wrist and hand including:
a wrist portion of the splint including a moveable closure configured to secure a wrist of the patient to the splint;
a hand portion of the splint rigidly coupled to the wrist portion, the hand portion including a hand portion that is shaped to receive a palm of the patient's hand;
an inflatable bladder coupled to the hand portion, the inflatable bladder including an input to the inflatable bladder;
a pump configured to generate a gas at an output of the pump;
a tube having a first opening coupled to the output of the pump and having a second opening coupled to the input to the inflatable bladder; and
a processor circuit configured to activate the pump to inflate the inflatable bladder responsive to a first input to the processor circuit and configured to deflate the inflatable bladder responsive to a second input to the processor circuit.

15. The system according to claim 12 further comprising a switch coupled to the tube, wherein the processor circuit is configured to detect an increase in pressure in the tube responsive to compressive force on the inflatable bladder while inflated as the second input to the processor circuit.

16. The system according to claim 12 further comprising a switch coupled to the tube, wherein the processor circuit is configured to deflate the inflatable bladder responsive to detecting a pre-programmed pressure level in the tube during inflation.
